(19) United States
(12) Patent Application Publication
(10) Pub. No.: US 2024/0419702 A1
(43) Pub. Date: Dec. 19, 2024

(54) METHODS AND SYSTEMS FOR AUTOMATIC APPEAL AUTHORIZATION USING MACHINE LEARNING ALGORITHM

(71) Applicant: Express Scripts Strategic Development, Inc., St. Louis, MO (US)

(72) Inventors: Malla Reddy Adaboina, Edison, NJ (US); Ramandeep Kaur, Waldwick, NJ (US)

(21) Appl. No.: 18/817,441

(22) Filed: Aug. 28, 2024

Related U.S. Application Data
(63) Continuation-in-part of application No. 18/082,745, filed on Dec. 16, 2022.

(51) Int. Cl. G06F 16/332; G06F 16/33; G06Q 40/08

(52) U.S. Cl. CPC G06F 16/3329; G06F 16/3347; G06Q 40/08

(57) ABSTRACT

Methods and systems for selecting a deep learning/machine learning model are described. In one embodiment, a plurality of models are optimized by addressing class imbalances among a first subset of records, each of the plurality of models are trained using the first subset of records, each of the plurality of models are implemented to predict a value of respective known target columns in each of a second subset of records, respective success rates for each of the plurality of models at predicting the respective known target columns for each of the second subset of records are determined, a first of the plurality of models having a highest success rate is selected, and the first of the plurality of models is implemented to decide a target column of a received authorization request based on predictor columns in the received authorization request.

### CROSS-REFERENCE TO RELATED APPLICATIONS

This application is also a continuation-in-part application claiming priority to U.S. application Ser. No. 18/082,745, entitled “Method and Systems for Automatic Authorization Using Machine Learning Algorithm”. The entire disclosure, including the specification and drawings, of U.S. application Ser. No. 18/082,745 is also incorporated herein by reference.

### FIELD

The present disclosure relates generally to the technical field of artificial intelligence/machine learning. In a specific example, the present disclosure may relate to using an artificial intelligence and/or machine learning model to make decisions regarding an authorization status.

### BACKGROUND

Authorizing access to a protected asset conventionally requires human oversight, intervention, or decision-making. For example, when a patient is prescribed a prescription drug, a pharmacy, such as a high-volume or mail-order pharmacy, may not dispense the prescription drug without prior authorization from a health plan provider, such as an insurance company or a pharmacy benefit manager, showing that the health plan provider will cover costs of the prescribed medication. Typically, such authorization occurs as a human reviewing a prescription claim, reviewing the patient's medical history, reviewing the patient's insurance coverage and/or financial situation, determining whether an alternative drug exists to fulfill the prescription, and approving or denying the claim. In some situations, the authorization process may involve the health provider negotiating a lower price with the drug manufacturer if the patient cannot afford the drug. A similar process occurs for other medical insurance claims related to medical procedures or care.

The approval process typically includes the following events: 1) a healthcare provider submits a prior authorization request to the patient's insurance company before performing a specific medical service, procedure, or prescribing a medication; 2) the insurance company decides whether to authorize or deny the prior authorization request, and 3) if the prior authorization is approved, the healthcare provider proceeds with the authorized medical service, procedure, or medication.

In some cases, the prior authorization request may be denied. In response to receiving a denial, patients may appeal the decision. Patient appeals sometimes occur when a new medical treatment arises or when conventional medical treatments are used in a new way. For example, some physicians have used Botox to treat migraine headaches. If a physician submitted a prior authorization to use Botox to treat migraines, a human appeals reviewer might deny such a claim because Botox is typically only used to treat wrinkles and is typically considered an elective drug for vanity purposes. A patient or the physician could appeal this decision by submitting, for example, sufficient clinical research showing that Botox can mitigate or alleviate migraine headaches. In light of new facts and new research showing that Botox can successfully be used to treat migraines, the human reviewer would approve the use of Botox to treat migraines in response to the appeal.

Primarily, because a human oversees prior authorizations appeals and makes the appeal decision, the average time to decide an appeal can be approximately two months but can last longer. The reason appeals take so long to complete is because doctors typically submit a significant number of documents with the appeal request, such as numerous clinical research papers and journals, additional patient information showing the necessity for the treatment (e.g. all conventional treatments have failed), and even expert opinions. Due to this slow decision-making process, costs increase for patients, physicians, and pharmacies, and viable treatments may be delayed for patients needing care.

### BRIEF DESCRIPTION OF THE DRAWINGS

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a block diagram of an example benefit manager device that may be deployed within the system of FIG. 1, according to an example embodiment;

FIG. 5 is a block diagram of a flowchart illustrating methods for training and selecting deep learning and/or machine learning models for predicting prior authorization status values, according to an example embodiment;

FIG. 6 is a block diagram of a flowchart illustrating methods for predicting prior authorization status values, according to an example embodiment;

FIG. 7 is a block diagram of an example benefit manager device that may be deployed within the system of FIG. 1, according to an example embodiment;

FIG. 8 is a block diagram of a flowchart illustrating methods for predicting prior authorization appeal status values, according to an example embodiment;

FIG. 9 is a block diagram of an example patient management platform that may be deployed within the system of FIG. 1, according to some examples; and

FIG. 10 is a functional block diagram of an example neural network that can be used for the model inference engine or other functions (e.g., engines) as described herein to produce various predictive models.

### DETAILED DESCRIPTION

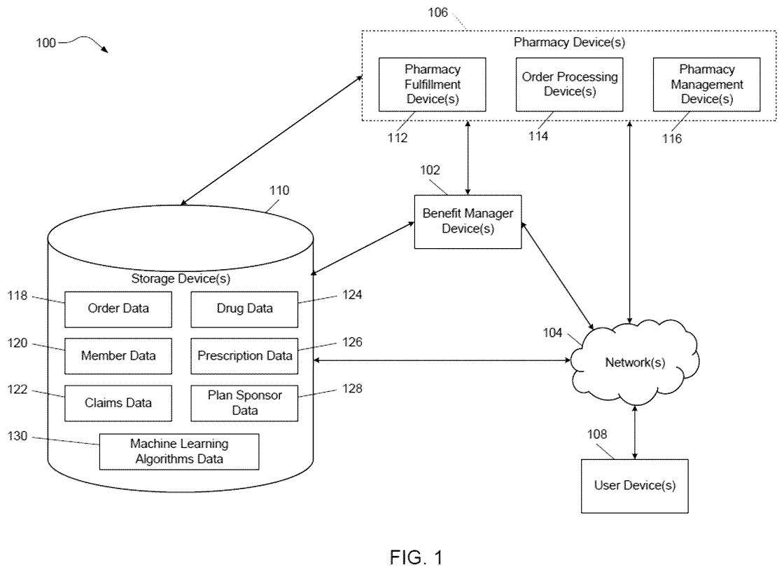

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104. The system 100 may also include a storage device 110.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long-term care benefit, a nursing home benefit, etc. The PBM may, in addition to its PBM operations, operate one or more pharmacies. The pharmacies may be retail pharmacies, mail order pharmacies, etc.

In some embodiments, the benefit manager device 102 can include one or more servers, supercomputers, or other computing device(s) configured to implement artificial intelligence models, such as a feed-forward neural network, a convolutional neural network, a recurrent neural network, a Random Forest model, a Naïve Bayes model, a decision trees model, a logistic regression model, generative artificial intelligence large language model (“LLM”), or any other deep learning and/or machine learning artificial intelligence model. In some embodiments, the benefit manager device 102 can include a group of computers, such as a neural network configured to implement artificial intelligence deep learning and/or machine learning models.

Some of the operations of the PBM that operates the benefit manager device 102 may include the following activities and processes. A member (or a person on behalf of the member) of a pharmacy benefit plan may obtain a prescription from a physician, and the member may seek to receive a prescription drug from a pharmacy. The prescription drug may require prior authorization from a health plan provider prior to dispensing the drug, and the PBM may request prior authorization. Upon receiving authorization approval, the member may obtain the prescription drug. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device 108, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in the storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member, receiving authorization approval, and dispensing the prescription drug to the member, the pharmacy submits a claim to the PBM for the prescription drug, which may require prior authorization. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. The PBM may also perform automatic prior authorization using deep learning and or machine learning with the claim submission, which may include determining whether to cover some or all of the costs related to dispensing the prescription drug, determining whether any cheaper alternative drugs may substitute for the prescription drug, determining whether the member can afford the costs to receive the prescription drug, and negotiating a lower price for the prescription drug on behalf of the member when the member cannot afford the full-price of the prescription drug. Further, the PBM may provide a response to the pharmacy (for example, the pharmacy system 100) following performance of at least some of the aforementioned operations, which many include approval or denial of the claim as a result of performing prior authorization.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, plan sponsor data 128, and/or deep learning/machine learning model data 130. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the use of the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications. The drug data 124 may further include an indicator specifying whether prior authorization is required before dispensing each drug.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

Furthermore, the deep learning/machine learning algorithms data 130 can include code or instructions necessary to implement each of multiple neural network models and/or machine learning models and/or large language models. The code or instructions can be implemented by one or more processors of the benefit manager device 102 or the pharmacy device 106. Each of the multiple neural network models and/or machine learning models can make a prediction for prior authorization status based on one or more factors (e.g. patient age, patient gender, prescribed drug, patient medical conditions), a prediction process that can be performed each time prior authorization is requested. Each of the multiple neural network models and/or machine learning models can include code or instructions to learn from a training dataset, as would be understood by one having ordinary skill in the art. Each of the multiple algorithms can change or adapt the code or instructions based on learning from the training dataset and make predictions according to the changed code or instructions. Additionally, some or all of the multiple algorithms can make predictions for an appeal of a denied prior authorization request. According to an exemplary embodiment, the multiple machine learning models can include the Random Forest machine learning algorithm, the K-Neighbor machine learning algorithm, the Gaussian Naïve Bayes machine learning algorithm, a decision tree algorithm, a logistical regression model, a generative artificial intelligence LLM model, and the SGD machine learning algorithm. According to an exemplary embodiment, the multiple neural network models can include a feed-forward neural network, a convolutional neural network, and a recurrent neural network.

In some embodiments, each of the multiple neural network models and/or machine learning algorithms can make a prediction as to prior authorization status (e.g., “approved” or “denied”). By predicting the prior authorization status, each of the multiple neural network models and/or machine learning algorithms can automatically authorize prescription or medical procedure claims, thereby saving time for physicians, pharmacies, and patients in filling a prescription.

In some embodiments, one or more of the multiple neural network models and/or machine learning algorithms can approve or deny an appeal. By evaluating prior authorization appeals, one or more of the multiple neural network models, generative artificial intelligence LLM, and/or machine learning algorithms can determine appeals, thereby resulting in an appeal decision far sooner than conventional methods. A significant amount of documentation supporting an appeal may accompany the appeal submission. For example, the documents accompanying the appeal submission may include updated medical records, clinical notes, peer-reviewed studies, or expert opinions. One or more of the neural network models, generative artificial intelligence, and/or machine learning algorithms can review the submitted documentation and decide an appeal based on the reviewed documentation in significantly less time than a human.

Each of the multiple neural network models and/or machine learning models can make predictions as to prior authorization status for a prescription using factors, such as patient age, patient gender, which drug has been prescribed, existing health conditions of the patient, the state where the patient lives, channel by which the prescription was submitted (e.g., electronic, in-person, etc.), patient height and weight, current medications taken by the patient, and patient race. Different factors can be used to make prior authorization status predictions, and the factors and number of factors used to make prior authorization status predictions can depend on which factors best lead to prediction accuracy. Prediction accuracy can further consider confusion metrics, such as the number of false positives and false negatives made in inaccurate predictions (with either false positives or false negatives being a more preferable result). Each of the multiple neural networks and/or machine learning models can learn and improve prediction success using a training dataset by factoring all or some of the factors listed above. Furthermore, after learning from the training dataset, each of the multiple neural networks and/or machine learning models can make predictions for a particular prescription based on all or some of the factors.

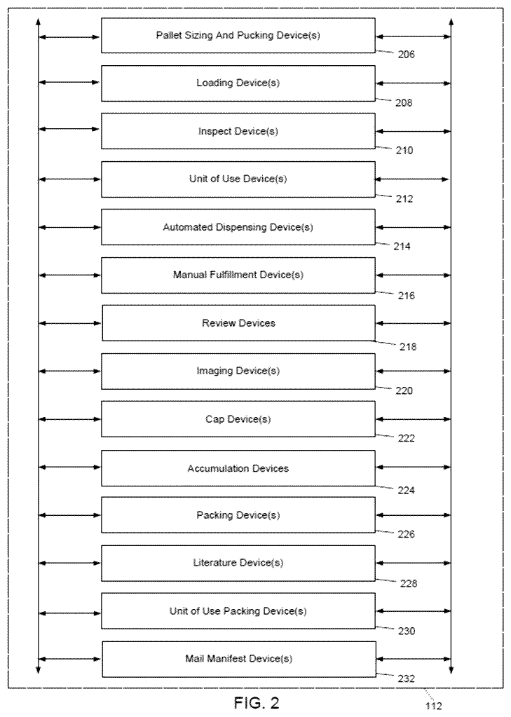

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

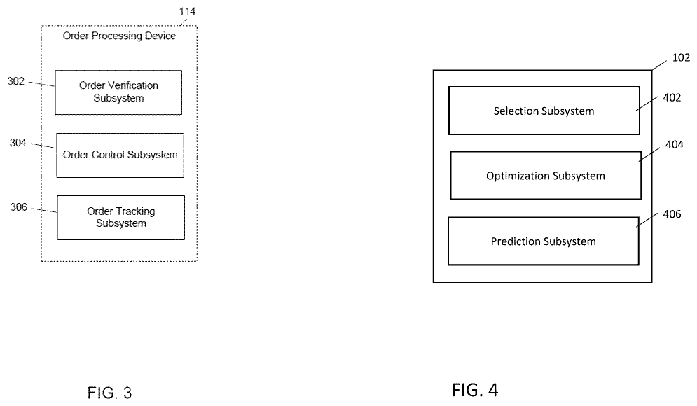

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may include order related components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

FIG. 4 illustrates the benefit manager device 102, according to an example embodiment. The benefit manager device 102 may be deployed in the system 100, or may otherwise be used. The benefit manager device 102 may include a selection subsystem 402, an optimization subsystem 404, and a prediction subsystem 406.

According to an exemplary embodiment, the selection subsystem 402 can determine which of the multiple deep learning and/or machine learning models is the most accurate for making decisions. In some embodiments, the selection subsystem 402 can determine which of the multiple deep learning and/or machine learning models is the most accurate for making prior authorization decisions for prescription drugs or other medical claims. The selection subsystem 402 can cause each of the multiple deep learning/machine learning models to predict prior authorization status (e.g., approval or denial) for each record in the test data. After running each of the multiple deep learning/machine learning models to predict prior authorization status for each record in the test data, the selection subsystem 402 can determine which of the multiple deep learning and/or machine learning models is the best model for making decisions. In some embodiments, the selection subsystem 402 can determine which of the multiple deep learning and/or machine learning models is the most accurate for making decisions and which of the multiple deep learning and/or machine learning models scores best in confusion metrics.

According to an exemplary embodiment, prediction accuracy can measure whether one of the multiple deep learning and/or machine learning models correctly predicted the status of the prior authorization request (e.g., correctly found that the request should be approved or denied) when applying the one of the multiple deep learning and/or machine learning models to historical data (i.e. historical records). According to an exemplary embodiment, the confusion metrics can evaluate whether the number of incorrect predictions included more false positives or more false negatives. In some embodiments, such as the prior authorization embodiment, a false positive can be a more desirable inaccurate finding than a false negative. In another embodiment, a false negative can be a more desirable inaccurate finding than a false positive. For example, the reason a false positive may be more desirable than a false negative is because a false negative is likely to lead to a member appeal, which can require significant time and resources from authorization staff and technology. Thus, the “best” model, as determined by the selection subsystem 402 may be a less accurate model that scores better in the confusion metrics. For example, a model that accurately predicts 90% of the prior authorization statuses but most of the inaccurate predictions are false negatives may be a “worse” model than a model that accurately predicts 89% of the prior authorization statuses but most of the inaccurate predictions are false positives. In this way, the selection subsystem 402 can consider both accuracy rate and confusion metrics in selecting a model for predicting decisions such as prior authorization statuses. In another embodiment, confusion metrics can calculate sensitivity and specificity of the model, which can measure the performance of the model that predicts some kind of classification. “Sensitivity” can describe a true positive rate of the model, where true positive rate means total number of true positive predictions (i.e., (the number of true positive predictions)/(the number of true positive predictions+number of false negative predictions)). For example, if the “sensitivity” is 90%, that means that when the model predicts 100 denials, 90 of the 100 denials can be true denials, and 10 of the 100 denials can be false denials or false negatives. Similarly, “specificity” can define the true negative rate of the model. True negative rate means the total number of true negative predictions (i.e., (the number of true negative predictions)/(the number of true negative predictions+number of false positive predictions)). For example, if the “specificity” is 90%, that means that when the model predicts 100 approvals, 90 of the 100 approvals can be true approvals and 10 of the 100 approvals can be false approvals or false positives.

According to an exemplary embodiment, the optimization subsystem 404 can train, customize, and optimize the multiple deep learning and/or machine learning models. For example, the optimization subsystem 404 can gather records involving prescription claims (e.g. the claims data 122). In some embodiments, the gathered records can include historical claim data related to a specific prescription drug or numerous prescription drugs accumulated over a period of time (e.g., a year, a month, a decade). After gathering the records, the optimization subsystem 404 can divide the records into, for example, three subsets: a first subset for training, a second subset for testing, and a third subset for validating. Subsequently, the optimization subsystem 404 can select a first of the multiple deep learning and/or machine learning models and train the first of the multiple deep learning and/or machine learning models using the first subset of the records. According to an exemplary embodiment, training the first of the multiple deep learning and/or machine learning models can include the first of the multiple deep learning and/or machine learning models analyzing each record to determine the known authorization status and a relationship between the known authorization status and factors (e.g. age, prescribed drug, etc.). In some embodiments, training the first of the multiple deep learning and/or machine learning models can further include determining a correlation coefficient of each of the factors and the authorization status. The result of determining a correlation coefficient of each of the factors can include disregarding some of the factors as irrelevant to the prediction process and also weighing more heavily or less heavily some of the factors in the prediction process. However, training the first of the multiple deep learning and/or machine learning models can include additional processes.

In addition to training the first of the multiple deep learning and/or machine learning models, the optimization subsystem 404 can address class imbalances among the factors in predicting authorization status. In some embodiments, some artificial intelligence models may ignore, and in turn, have poor performance on the minority class or minority classes. To prevent class imbalance, the optimization subsystem 404 can employ synthetic minority oversampling technique (“SMOTE”), as would be known to those skilled in the art. SMOTE can effectively address the imbalance by creating synthetic datapoints corresponding to the minority class. For example, heart disease happens in various age groups, communities, and lifestyles, and the gathered records may not have an equal amount of data for all the various age groups, communities, and lifestyles where heart disease occurs. SMOTE can create synthetic data for minority classes in the heart disease example to address any class imbalances. Moreover, the optimization subsystem 404 can also use categorical embedding for any categorical predictors, such as member's state or drug name.

The optimization subsystem 404 can further receive user feedback to optimize the first of the multiple deep learning and/or machine learning models. The optimization subsystem 404 can receive user feedback after the first of the multiple deep learning and/or machine learning models begins predicting authorization statuses, and if the optimization subsystem 404 incorrectly predicts an authorization status, a user can provide negative feedback indicating that the decision was incorrect. In response, the optimization subsystem 404 can learn from the feedback and further optimize the first of the multiple deep learning and/or machine learning models. In some embodiments, user feedback can also indicate when the first of the multiple deep learning and/or machine learning models correctly predicted an authorization status, and the optimization subsystem 404 can also learn from positive feedback and further optimize the first of the multiple deep learning and/or machine learning models.

Further still, the optimization subsystem 404 can additionally optimize the first of the multiple deep learning and/or machine learning models by determining layers of the first of the multiple deep learning and/or machine learning models, editing error functions of the first of the multiple deep learning and/or machine learning models, editing epochs of the first of the multiple deep learning and/or machine learning models, editing the depth of the first of the multiple deep learning and/or machine learning models, and other customization methods as would be understood by those having skill in the art. For example, customization may include editing average or maximum pooling, or adjusting other parameters of the first of the multiple deep learning and/or machine learning models. Customization or optimization may also include customizing the number of network layers. These customizations and optimizations may depend on the first subset of the records or all of the gathered records.

According to an exemplary embodiment, pooling layers can help down sampling feature maps by summarizing the presence of features of the feature map, which can include two common methods: average pooling and max pooling. In some embodiments, average pooling can summarize average presence of the features, and max pooling can determine the most activated presence of the features. Additionally, average and max pooling can include cross entropy, which can be a default loss function for a binary classification problem where target value or 0 or 1. In addition, the systems and methods can use categorical cross entropy as a loss function, which has a soft max activation function, and cross entropy loss can output the probability over C classes for each target column.

As described above, the optimization subsystem 404 can determine the number of nodes in each layer, activation functions on each layer, like “relu” or “sigmoid” or “softmax”. Compiling the model with a proper loss function and optimizers like “rmsdrop” can be tuned to improve performance of the model. Adding pooling layers in between the network layers can also improve model performance.

After training, the optimization subsystem 404 can implement the first of the multiple deep learning and/or machine learning models, as trained, on the second subset to measure the accuracy after each cycle. Also, the optimization subsystem 404 can implement the first of the multiple deep learning and/or machine learning models, as trained, on the third subset to test the first of the multiple deep learning and/or machine learning models at predicting prior authorization statuses. The optimization subsystem 404 can pass the results of implementing first of the multiple deep learning and/or machine learning models on the second and third subsets to the selection subsystem 402, and the selection subsystem 402 can determine the success rate of the first of the multiple deep learning and/or machine learning models at predicting prior authorization statuses based on prediction accuracy and confusion metrics, as described above.

The optimization subsystem 404 can repeat the process described above using a second of the multiple deep learning and/or machine learning models, namely the optimization subsystem 404 can train the second of the multiple deep learning and/or machine learning models using the first subset of the gathered records, the optimization subsystem 404 can implement the second of the multiple deep learning and/or machine learning models to validate and test how the second of the multiple deep learning and/or machine learning models predicts prior authorization statuses, and the selection subsystem 402 can determine the success rate of the second of the multiple deep learning and/or machine learning models. This process repeats until the optimization subsystem 404 optimizes each of the multiple deep learning and/or machine learning models, and the selection subsystem 402 determines the success rate for each of the multiple deep learning and/or machine learning models. Finally, the deep learning and/or machine learning model having the highest success rate is chosen and passed to the prediction subsystem 406. The process of selecting a machine learning model can repeat periodically (e.g. once a week, each time a prior authorization request is received, or once at initialization.

After the selection subsystem 402 determines which of the multiple machine learning algorithms has the highest success rate for a given dataset, the prediction subsystem 406 can use the selected model in predicting prior authorization statuses. The prediction subsystem 406 can receive a prior authorization request, consider the factors included in the received prior authorization request, invoke the deep learning and/or machine learning model selected by the selection subsystem 402, and authorize or deny the prior authorization request according to the deep learning and/or machine learning model selected by the selection subsystem 402 and as configured and optimized by the optimization subsystem 404.

In some embodiments, the modules of the prediction subsystem 406 may be distributed so that some modules are deployed in the benefit manager device 102 and some modules are deployed in the pharmacy device 106. In one embodiment, the modules are deployed in memory and executed by a processor coupled to the memory.

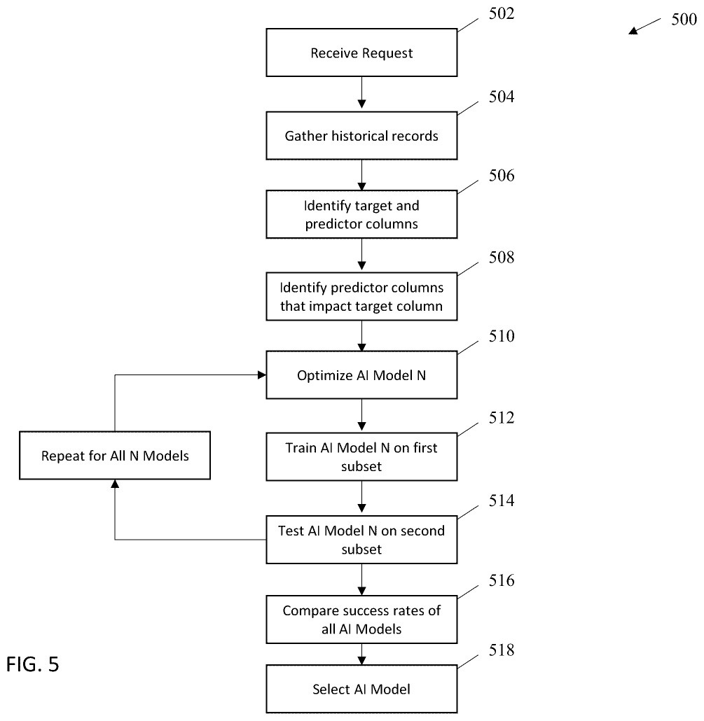

FIG. 5 illustrates a method 500 for selecting one of multiple deep learning and/or machine learning models according to an example embodiment. The method 500 may be performed by the benefit manager device 102, partially by the benefit manager device 102 and partially by the pharmacy device 106, or may be otherwise performed. For the sake of simplicity, the benefit manager device 102 will be described as performing the steps of the method 500, but the embodiments described herein are not so limited.

According to an exemplary embodiment, the benefit manager device 102 can receive a prior authorization request in step 502. In some embodiments, the prior authorization request can be related to a prescription drug prescribed by a doctor to a patient or member. The prior authorization request can include various factors about the request, including but not limited to patient age, patient gender, which drug has been prescribed, existing health conditions of the patient, the state where the patient lives, channel by which the prescription was submitted (e.g., electronic, in-person, etc.), patient height and weight, current medications taken by the patient, and patient race. The prior authorization request may include some, all, or more than the factors listed above.

In step 504, the benefit manager device 102 can gather historical claim records (i.e. historical records). In some embodiments, the gathered historical claim records can be related to the prescription drug identified in the prior authorization request. Subsequently, the benefit manager device 102 can identify a target column and predictor columns in step 506. For example, the benefit manager can identify the target column as the prior authorization status (“approved” or “denied”) and the predictor columns as the factors included in the prior authorization request. After identifying the target and predictor columns, the benefit manager device 102 can identify which factors/predictor columns impact the target column in step 508, which can include the benefit manager device 102 determining that some factors are irrelevant to predicting the target column, and/or the benefit manager device 102 determining a correlation coefficient on each predictor column in predicting the target column. Step 508 can result in a final set of predictor variables and the weight given to each predictor variable in deciding the target column. For example, a patient's existing health conditions may weigh heavily in denying a prior authorization request. As an example, a woman seeking an intrauterine birth control device may be denied, regardless of age, weight, race, etc., if the woman has uterine fibroids. In this example, the existence of uterine fibroids would result in a claim denial, and instead the benefit manager device 102 may substitute the intrauterine birth control device with birth control pills.

Subsequently, the benefit manager device 102 can select a first of multiple deep learning/machine learning models and optimize the first of multiple deep learning/machine learning models in step 510. Optimizing the first of multiple deep learning/machine learning models in step 510 can include using SMOTE to balance class imbalances, performing categorical embedding, selecting an error function for the first of multiple deep learning/machine learning models, determining layers, epochs, and min/max pooling for the first of multiple deep learning/machine learning models, etc. After optimizing the first of multiple deep learning/machine learning models, the benefit manager device 102 can train the first of multiple deep learning/machine learning models using a first subset of historical data in step 512. After training, the benefit manager device 102 can test prediction accuracy of the trained first of multiple deep learning/machine learning models using a second subset of the historical data in step 514. The benefit manager device can repeat steps 510-514 for each of the multiple deep learning/machine learning models.

After testing each of the multiple deep learning/machine learning models, in step 516, the benefit manager device 102 can compare success rates for each of the multiple deep learning/machine learning models to determine which of the deep learning/machine learning models had the highest success rate in predicting the target column. According to an exemplary embodiment, the benefit manager device 102 can consider both prediction accuracy and confusion metrics when determining which of the deep learning/machine learning models had the highest success rate in predicting the target column. Subsequently to comparing success rates for each of the multiple deep learning/machine learning models, the benefit manager device 102 can select one of the multiple deep learning/machine learning models having the highest success rate for predicting the received prior authorization request in step 518.

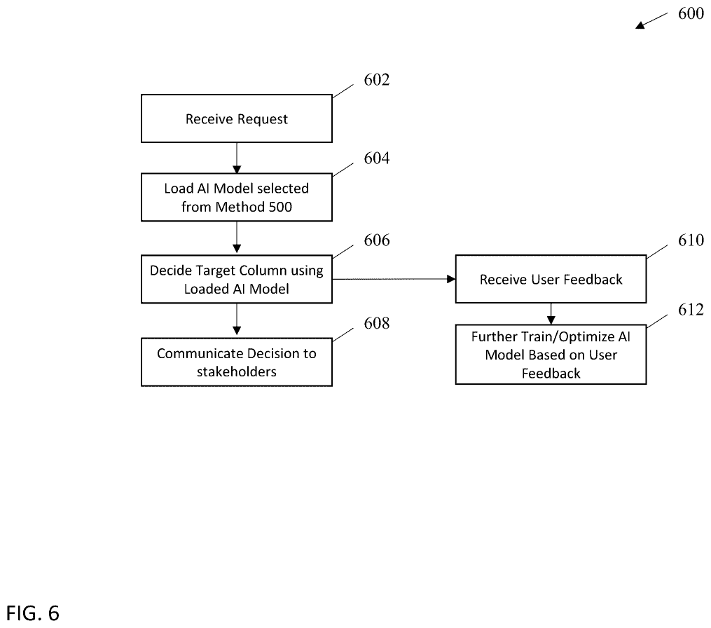

FIG. 6 illustrates a method 600 for predicting a target column according to an example embodiment. The method 600 may be performed by the benefit manager device 102, partially by the benefit manager device 102 and partially by the pharmacy device 106, or may be otherwise performed. For the sake of simplicity, the benefit manager device 102 will be described as performing the steps of the method 600, but the embodiments described herein are not so limited.

Although not illustrated, several steps are usually performed before or simultaneously with the illustrated steps of the method 600. For example, a patient can visit a doctor, the doctor can write a prescription, and the prescription can be provided to a pharmacy. After receiving the prescription, the pharmacy may contact a PBM or other benefit providing entity (e.g., insurer) to conduct prior authorization and determine other prescription benefits. The method 600 can invoke the selected deep learning/machine learning model, as determined by the method 500, to make the prior authorization decision without human involvement.

The benefit manager device 102 can receive a prior authorization request and accept input parameters (i.e. factors) for predictions in step 602. Furthermore, the benefit manager device 102 can load one of the multiple deep learning/machine learning models to predict the target column in step 604. In some embodiments, the deep learning/machine learning model loaded can be the deep learning/machine learning model having the highest success rate, as determined in the method 500, described above. Furthermore, the benefit manager device 102 can decide the target column (e.g., prior authorization status) in step 606 using the loaded deep learning/machine learning model, and the benefit manager device 102 can communicate the target column decision to interested stakeholders, such as the doctor, the pharmacy, and the patient, in step 608

In addition, in step 610, the benefit manager device 102 can receive feedback from a user regarding the decision made in step 606. The feedback can either be positive feedback (indicating the benefit manager device 102 correctly decided the target column) or negative feedback (indicating the benefit manager device 102 incorrectly decided the target column). In response to the feedback, the benefit manager device 102 can further train/optimize the loaded deep learning/machine learning model in step 612.

In view of the following, complicated decisions that conventionally took days, weeks, or even months can be decided using the systems and methods described herein in mere seconds. Because the systems and methods can decide prior authorization requests many times faster than conventional methods, the systems and methods improve the functioning of technology used for conventional prior authorization methods. In addition, the systems and methods described herein are more accurate at making decisions that a human being is, as shown through empirical testing data. After testing the systems and methods on a particular drug (e.g. Diclofenac), the most accurate deep learning/machine learning model was found to accurately predict prior authorization system at a rate of 90% before receiving any feedback from users and after only an initial training phase. This 90% accuracy is higher than conventional methods because more than 10% of conventional prior authorization decisions were overturned on appeal. Thus, not only do the systems and methods disclosed herein improve decision-making speed, the systems and methods disclosed herein improve decision-making accuracy. Thus, the systems and methods improve the functioning of computer resources to make prior authorization decisions and also provide an improvement to another technical field.

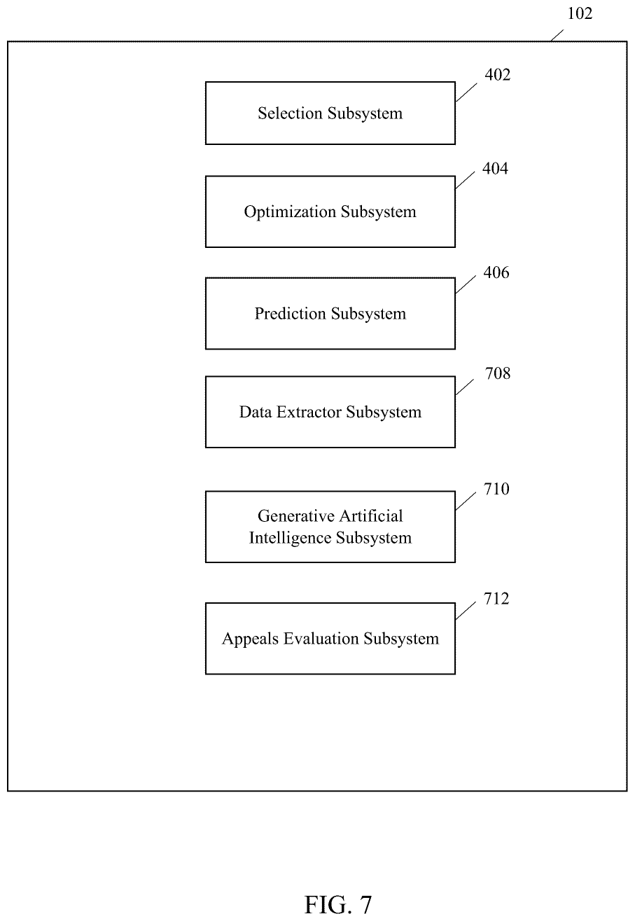

FIG. 7 illustrates the benefit manager device 102, according to another example embodiment. The benefit manager device 102 may be deployed in the system 100, or may otherwise be used. The benefit manager device 102 may include the selection subsystem 402 described above with regard to FIG. 4, the optimization subsystem 404 described above with regard to FIG. 4, the prediction subsystem 406 described above with regard to FIG. 4, a data extractor subsystem 708, a generative artificial intelligence subsystem 710, and an appeals evaluation subsystem 712.

According to an exemplary embodiment, the selection subsystem 402, the optimization subsystem 404, and the prediction subsystem 406 can operate as described above with reference to FIGS. 4-6. The selection subsystem 402, the optimization subsystem 404, and the prediction subsystem 406 may be implemented with the data extractor subsystem 708, the generative artificial intelligence subsystem 710, and the appeals evaluation subsystem 712 in an embodiment involving prior authorization appeals. According to an exemplary embodiment, the selection subsystem 402, the optimization subsystem 404, and the prediction subsystem 406 can be used in conjunction with or after operations performed by the data extractor subsystem 708, the generative artificial intelligence subsystem 710, and the appeals evaluation subsystem 712. In the embodiment illustrated in FIG. 7, the data extractor subsystem 708, the generative artificial intelligence subsystem 710, and the appeals evaluation subsystem 712 may be called after the prediction subsystem 406 denies a prior authorization request, and a patient seeks to appeal the denial.

The data extractor subsystem 708 can receive an appeal request and data accompanying the appeals request. In some embodiments, the data accompanying the appeals request can include documentation supporting the appeal and supporting that a previous decision to deny a prior authorization request, either by the prediction subsystem 406 or by a human, was made in error. The documentation accompanying the appeals request can include some or all of updated medical records for a patient associated with the denied prior authorization request, clinical notes, peer reviewed studies, and expert opinions.

According to an exemplary embodiment, the prediction subsystem 406 or a human may have denied a prior authorization request because a medical treatment is not approved as part of an official policy of an insurance company or a drug benefit plan. As an example, the drug Botox may be approved as an official policy of the insurance company as a treatment for temporomandibular joint (“TMJ”) pain, but Botox may not be approved as an official policy as a treatment for migraine headaches. If a patient submitted a prior authorization request for insurance or a drug benefit plan to cover the cost of Botox as a treatment for migraine headaches, the prediction subsystem 406 may deny the prior authorization request because Botox is not an approved treatment for migraine headaches according to official policy. However, policies can change in response to new research in the medical field or compelling data suggesting a new or unconventional treatment should be attempted for some patients. As such, the documentation supporting an appeal may include clinical research or medical journals supporting the appeal, a more extensive patient medical history chart, expert opinions, or added detail about the patient's condition from a physician. In the example given, the documentation can include clinical research or medical journals showing that Botox is particularly effective at alleviating migraine headache pain for many similarly situated patients. The documentation may further show that many conventional methods for managing migraine pain have proven ineffective at alleviating migraine pain for this particular patient, and thus, unconventional methods should be attempted. In some embodiments, the amount of documentation and data provided with the appeal request is significant, and would take a human many hours or days to fully understand and review. Moreover, the submitted documentation may only be understandable to a person having a medical degree or a strong scientific background.

After receiving the appeal request and the associated documentation, the data extractor subsystem 708 can extract all the data from the documentation and create word embeddings of the data included in the documentation. In some embodiments, the data extractor subsystem 708 can create word embeddings by converting the data included in the documentation into vectors using natural language processing. The data extractor subsystem 708 can use natural language vectorization such that similar semantic information is stored close together as a vector in a vector database. In other words, similar semantic information is stored as a nearest neighbor in the vector database. As would be known to those having skill in the art, by vectorizing data and implementing word embedding, answers to questions are stored near question vectors in the vector database, and distance between vectors can assist the generative artificial intelligence LLM 710 in finding candidate vectors when searching a database. Thus, the data extractor subsystem 708 can extract data, embed the data as numerous vectors, and store the vectors in a vector database. The word embeddings stored as vectors can correspond to the number of features of the data, and the number of features can correspond to the number of dimensions in the high-dimensional vector database space.

In general, the associated documentation received with the appeal request can include medical records, clinical notes, peer-reviewed studies, or expert opinions. The data included in the associated documentation can include, previously answered questions (i.e., answers provided during the initial prior authorization request that was denied), prescriber information, patient information, previous claims made by the patient, the patient's medication history, medical test results, laboratory results, patient demographic information, the patient's existing medical conditions, the patient's age, a comorbidities, drug type, and policy information associated with the patient. In addition to receiving data with an appeals request, the data extractor subsystem 708 can obtain data from various databases, such as the storage device 110. The storage device 110 can store some of the information listed above (e.g., claims history, medication history, patient demographic information) and also other information, such as whether a drug has any alternatives.

In addition to receiving documentation and data with the appeal request, the data extractor subsystem 708 can further receive a set of questions. According to an exemplary embodiment, a pharmacist may generate the set of questions for each known drug or each known medical procedure for which a prior authorization has been requested. Continuing the example above, a pharmacist may generate a list of questions which help determine whether or not the insurance company or the PBM will cover the costs of Botox to treat migraine headaches. The list of questions may include “has the patient been diagnosed with chronic migraine pain?”; “how long has the patient been suffering from migraine pain?”; “has the patient been prescribed conventional migraine pain management medication, such as triptans or ditans?”; etc. A similar set of questions can exist for every drug or medical procedure for which a prior authorization can be submitted. After receiving the set of questions, the data extractor subsystem 708 can create word embeddings, or vectors, for each question in the set of questions. The set of questions may be referenced by determining the drug or medical procedure that is subject to the appeal or by referencing the denied prior authorization, such as a prior authorization claim number or the like.

In addition, the data extractor subsystem 708 can implement retrieval-augmented generation (“RAG”) for prompt engineering. The data extractor subsystem 708 may implement RAG for prompt engineering to provide relevant context to the generative artificial intelligence subsystem 710. The data extractor subsystem 708 can use RAG to ensure that the generative artificial intelligence subsystem 710 references authoritative knowledge outside a training dataset before generating a response. According to an exemplary embodiment, the authoritative knowledge can be the documents accompanying the appeal request. In this way, the data extractor subsystem 708 can use RAG to optimize the generative artificial intelligence subsystem 710 and extend the generative artificial intelligence subsystem 710 to specific domains without the need to retrain the generative artificial intelligence subsystem 710 each time new documents arrive with an appeal request. As such, the data extractor subsystem 708 can decrease hallucinations.

In order to implement RAG, the data extractor subsystem 708 can pull new information from the documents accompanying the appeal request, and then give the new information to the generative artificial intelligence subsystem 710 along with questions from the questionnaire associated with the requested drug or medical procedure in the form of prompts. Additionally, the data extractor subsystem 708 can generate a feature vector for each document provided with the appeal request to give each document a relevant context or value, or the data extractor subsystem can generate word embeddings for all of the data included in the documents accompanying the appeal request. Generating word embeddings can include storing the text data as vectors in a vector database.

After forming a vector database with the data included in the documents accompanying the appeal request, the data extractor subsystem 708 can pull the questions from the questionnaires related to the drug or medical procedure that is subject to the appeal (e.g., requesting that insurance pay for the drug Botox as a treatment for migraine headaches). The data extractor subsystem 708 can create word embeddings for the questions as well, which can involve generating feature vectors for the questions in the vector database.

RAG can further include searching within the vector database according to any searching system, such as approximate nearest neighbor (ANN), Euclidean distance, cosine similarity, or dot product. The data extractor subsystem 708 can also compute a feature vector for each question in the set of questions. Using the question's feature vector, the data extractor subsystem 708 can query a vector database to retrieve the most relevant documents or word embeddings to the question feature vector. The data extractor subsystem 708 can determine the most relevant documents or word embeddings in the vector database to the question's feature vector by searching for a predetermined number of nearest neighbor word vectors. The data extractor subsystem 708 can use cosine similarity to determine the predetermined number of nearest neighbor word vectors. In some embodiments, the predetermined number may be four, but other amounts of nearest neighbors are contemplated. However, the number of nearest neighbors should be sufficiently small enough to ensure that only relevant data is provided to the generative artificial intelligence subsystem while still casting a wide enough net to gather relevant text, which may come from more than one source (i.e., document). Once the data extractor subsystem 708 determines the predetermined number of nearest neighbor word vectors, the data extractor subsystem 708 concatenates the text contained in the predetermined number of nearest neighbor word vectors.

Additionally, the data extractor subsystem 708 can implement RAG to create a prompt to be provided to the generative artificial intelligence subsystem 710. The data extractor subsystem 708 can create the prompt using the combination of the question from the questionnaire and the concatenated text generated by finding the predetermined number of nearest neighbor word vectors. The RAG may be programmed with prompt engineering techniques to effectively communicate with the generative artificial intelligence subsystem 710 without hallucinations. The data extractor subsystem 708 can send the generated prompt to the generative artificial intelligence subsystem 710. The process of generating a prompt to be sent to the generative artificial intelligence subsystem 710 may occur for each question in the criteria (i.e., each question in the prior authorization questionnaire).

Moreover, additional prompt engineering can occur during set-up or creation of the data extractor subsystem 708. For example, the questions in the questionnaire may be sufficiently clear to a human pharmacist evaluating a prior authorization request, but the questions may be vaguely worded to an artificial intelligence algorithm, such as a large language model (LLM). As such, additional prompt engineering may occur such that questionnaire questions are reworded to be better understood by the LLM, such as the generative artificial intelligence subsystem 710. Structuring a question through prompt engineering may decrease hallucinations by the generative artificial intelligence subsystem 710.

The generative artificial intelligence subsystem 710 can implement a generative artificial intelligence algorithm. In some embodiments, the generative artificial intelligence algorithm can be a large language model. The generative artificial intelligence subsystem 710 can receive prompts from the data extractor subsystem 708 and output answers using the prompt, which can include the data included in the nearest neighbor vectors. The generative artificial intelligence subsystem 710 may be trained on a training dataset to provide answers to prompts. The additional data included in the prompt from the nearest neighbor vectors can add additional context and allow the generative artificial intelligence subsystem 710 to generate an answer to the prompt that is highly relevant and considers the relevant data (as determined by RAG and ANN) included in the documents accompanying the appeal request. The generative artificial intelligence subsystem 710 can save the answers by the LLM to each prompt in a database. The answers to each prompt may be saved with the questions from the questionnaire.

The generative artificial intelligence subsystem 710 can generate answers for every prompt provided and save the answers to the database. After the generative artificial intelligence subsystem 710 answers all the prompts, the appeals evaluation subsystem 712 can provide all the answers generated by the generative artificial intelligence subsystem 710 to the prediction subsystem 406 as an additional column to the machine learning module selected by the selection subsystem 402. In some embodiments, the benefit manager device 102 can perform the methods 500 and 600 described above with reference to FIGS. 5 and 6 to select, train, and optimize one of a plurality of machine learning algorithms, and then implement the selected, trained, and optimized machine learning algorithm to predict the status of a prior authorization appeal. Alternatively, the selection method 500 of FIG. 5 may be omitted for the appeals process because the selection subsystem 402 has already selected, trained, and optimized a machine learning algorithm during the prior authorization process that resulted in a prior authorization denial. The prediction subsystem 406 can re-predict the prior authorization status using the original data and the answers from the generative artificial intelligence subsystem 710. For some questions, the answers from the generative artificial intelligence subsystem 710 may replace data considered during the initial prior authorization prediction process that led to the appeal, and for some questions, the answers from the generative artificial intelligence subsystem 710 may append data considered during the initial prior authorization prediction process that led to the appeal. Using the new data, the prediction subsystem 406 can generate a decision on the appeal, which may or may not be different from the initial denial of the prior authorization request.

The prediction subsystem 406 can rely on data points to make decisions. According to an embodiment, the prediction subsystem 406 can weight some data points more heavily than others. Indeed, the prediction subsystem 406 can disregard some data points entirely to prevent bias by the machine learning algorithms. For example, the prediction subsystem 406 can disregard some patient demographics, such race, geographic location (city, state, country) educational qualifications, or income level. The prediction subsystem 406 can also disregard other irrelevant data points, such as testing location or laboratory location where a medical test occurred geographically (however, the prediction subsystem 406 may consider or weigh less heavily lab results from laboratories outside of the United States). On the other hand, the prediction subsystem 406 can consider other drug-related data points, such as alternative drugs, policy coverage, drug type, and other patient-related data points, such as previous claims made by the patient, a patient's existing medical conditions, a patient's age, gender, or past history with a drug, comorbidities, and diagnostic information. The prediction subsystem 406 can weight each of these data points more or less heavily, and the prediction subsystem 406 can change the weights in response to training or learning over time. The prediction subsystem 406 can consider alternative drugs by knowing available alternatives to determine if a requested drug is necessary or if there is a more cost-effective option. The prediction subsystem 406 can consider policy coverage to understand an insurance policy's coverage to determine if prior authorization is a requirement. The prediction subsystem 406 can consider drug type by considering whether a drug is a generic, and the prediction subsystem 406 may be more likely to approve a generic drug for coverage. The prediction subsystem 406 can consider previous claims to understand a patient's medical history and treatment patterns. The prediction subsystem 406 can consider the patient's existing medical conditions to determine if a drug is appropriate and necessary. The prediction subsystem 406 can consider a patient's age, gender, and past history to determine whether a prescribed dosage is appropriate. The prediction subsystem 406 can consider comorbidities to determine whether a prescribed drug might interact with any of the patient's medical conditions or other prescribed drugs, which may affect a drug's safety or efficacy. The prediction subsystem 406 can consider diagnostic information because some laboratory results can provide concrete evidence of the patient's condition and justify the need for some medications.

The appeals evaluation subsystem 712 can receive the decision from the prediction subsystem 406. If the prediction subsystem 406 approves the appeal and allows the prior authorization, the appeals evaluation subsystem 712 will grant the appeal and notify the patient and any interested physicians or medical providers. If the prediction subsystem 406 denies the appeal, the appeals evaluation subsystem 712 can pass the appeal to a human to review the appeal manually.

Because the machine learning algorithms implemented by the prediction subsystem 406 receive another column from the generative artificial intelligence subsystem 710, the machine learning algorithms implemented by the prediction subsystem 406 also become more intelligent, which can decrease the number of appeals necessary. For example, having learned from overturning a prior authorization denial through the appeals process, the machine learning algorithm can learn that additional data may indicate that similar a prior authorization request received subsequently should be approved. In addition, the prediction subsystem 406 can attach new weights to various predictor columns in response to successful appeals that overturn a previously denied claim decision by the prediction subsystem 406.

For example, after evaluating one or more appeals related to Botox for migraines, the prediction subsystem 406 (and the machine learning algorithm implemented by the prediction subsystem 406) may learn that some predictor columns should weight more heavily than others for criteria. For example, the predictor columns for all appeal decisions can include patient gender, residence state, patient age, patient weight, patient's existing health conditions, patient's current medications, patient's race, medical notes, reasons for the prior authorization denial, and appeals supporting documentation. The generative artificial intelligence subsystem 710 can apply weights to each of the predictor columns, and the generative artificial intelligence subsystem 710 can change the weighting values for each predictor columns as it learns. For example, the generative artificial intelligence subsystem 710 may find that the most important indicator for overturning a prior authorization denial for using Botox to treat migraine headaches is the existing health conditions predictor column, more specifically, if the existing health conditions predictor column indicates that the patient has suffered from migraine headaches for many years. Additionally, the added column from the generative artificial intelligence LLM subsystem 710 provides additional considerations that re-train the machine learning algorithms and improve their intelligence.

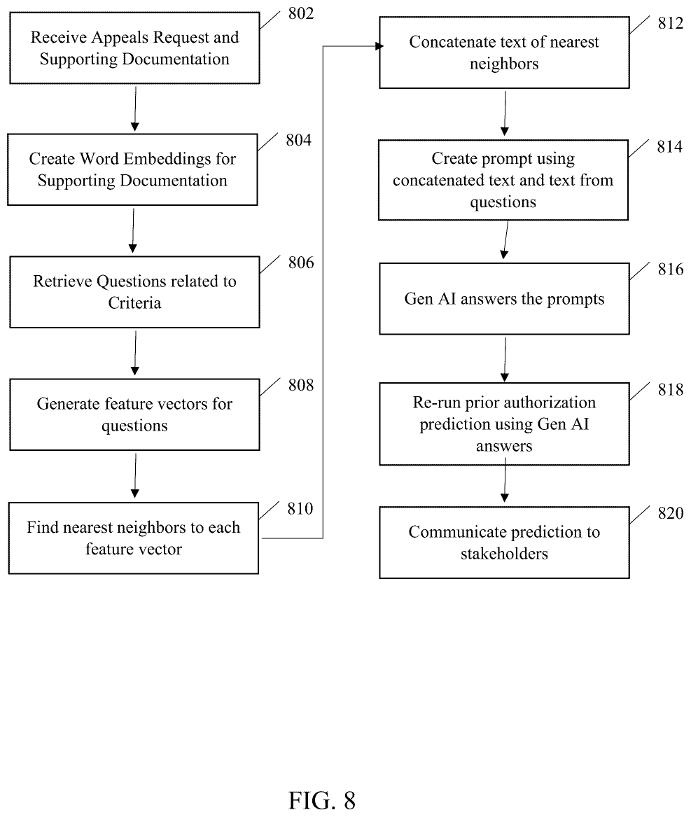

FIG. 8 illustrates a method 800 for predicting an appeal status according to an example embodiment. The method 800 may be performed by the benefit manager device 102, partially by the benefit manager device 102 and partially by the pharmacy device 106, or may be otherwise performed. For the sake of simplicity, the benefit manager device 102 will be described as performing the steps of the method 800, but the embodiments described herein are not so limited.

Although not illustrated, several steps are usually performed before or simultaneously with the illustrated steps of the method 800. For example, a patient previously submitted a prior authorization request for a prescription drug or medical procedure and the prior authorization request was denied. The method 800 can invoke the selected plurality of artificial intelligence/machine learning models, as determined by the method 500, to make the appeals decision without human involvement.

The method 800 can include the benefit manager device 102 receiving an appeal request and supporting documentation in step 802. In response to receiving the appeal request and the supporting documentation, the data extractor subsystem 708 can extract data in the supporting documentation and create word embeddings for the data in the supporting documentation in step 804. In some embodiments, creating the word embeddings can include converting the text into word vectors and saving the word vectors in a vector database according to a natural language vectorization technique such that similar semantic information is stored close together as a vector in a vector database. The data extractor subsystem 708 can pull questions related to criteria identified in the appeal request (e.g., the medical procedure or drug that is subject to the appeal) in step 806. In some embodiments, the data extractor subsystem 708 can reference a denied prior authorization, a decision which the appeal request is seeking to overturn. The denied prior authorization can include questions from a questionnaire used to determine whether to approve or deny a prior authorization request. In some embodiments, the data extractor subsystem 708 can also retrieve answers to the questions answered during the denied prior authorization.

Based on the questions related to the criteria (e.g., questions from the questionnaire), the data extractor subsystem 708 can generate feature vectors for each of the questions in step 808. Using the feature vectors for each of the questions generated in step 808, the data extractor subsystem 708 can find a predetermined number of nearest embeddings to the question feature vector in a vector space within the vector database in step 810. The data extractor subsystem 708 can concatenate text from the predetermined number of nearest embeddings to the question feature vector in step 812. In addition, the data extractor subsystem 708 can create a prompt for a large language model or other generative artificial intelligence large language model by combining the concatenated text and text from the question associated with the question feature vector in step 814. The data extractor subsystem 708 can implement RAG to perform the prompt engineering that results in the prompt created in step 814. The method 800 can perform this step for all questions in the questionnaire.

The generative artificial intelligence subsystem 710 can receive the prompt and answer the prompt in step 816. The appeals evaluation subsystem 712 can save each answer to each prompt generated by the generative artificial intelligence subsystem 710 in a database. After the generative artificial intelligence subsystem 710 generates an answer to every prompt created by the data extractor subsystem 708, the appeals evaluation subsystem 712 provides the answers generated by the generative artificial intelligence subsystem 710 to the prediction subsystem 406, and the prediction subsystem 406 re-runs the prior authorization prediction process by implementing a machine learning algorithm, as described above with reference to FIG. 6, using the answers generated by the generative artificial intelligence subsystem 710 in step 818. In some embodiments, the prediction subsystem 406 further references old data included in the prior authorization request as well as the answers generated by the generative artificial intelligence subsystem 710. The prediction subsystem 406 makes a prediction, and communicates the prediction to any stakeholders (e.g., patient, physician, insurance company, etc.) in step 820.

In some embodiments, the appeals evaluation subsystem 712 can call the selection subsystem 402 and the optimization subsystem 404 to again determine the best machine learning algorithm for the prediction subsystem 406 to use when re-running the priori authorization during the appeal process, according to the method 500 described with reference to FIG. 5. However, this process may be omitted, and the prediction subsystem 406 may implement the machine learning algorithm previously selected during the initial prior authorization that lead to the denial.

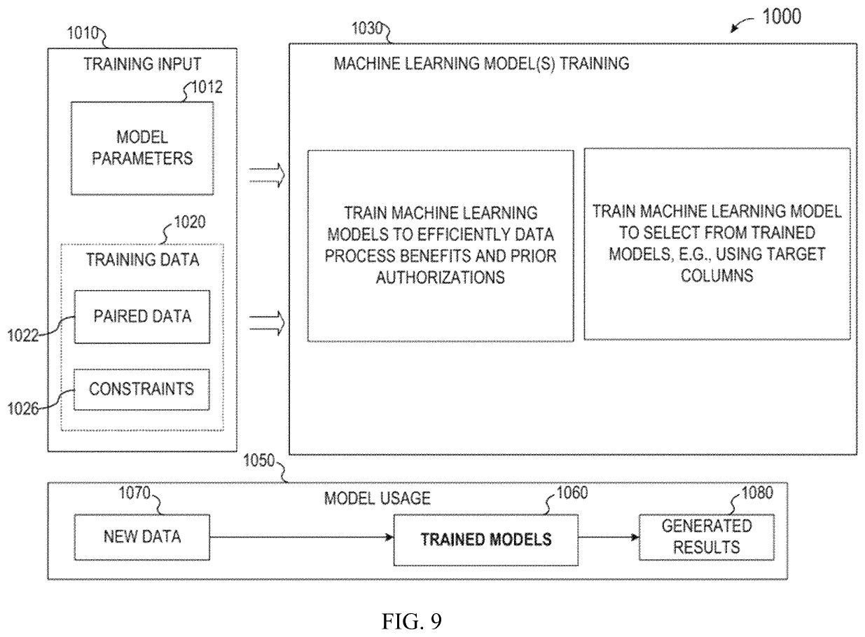

FIG. 9 is a block diagram of an example service of model platform 1000 that may be deployed within the system of FIG. 1, according to some examples. Training input 1010 includes model parameters 1012 and training data 1020 (e.g., data stored in storage 110) which may include paired training data sets 1022 (e.g., input-output training pairs) and constraints 1026. The model platform 1000 can be part of the benefit manager device, which can provide prior authorization functionality or prior authorization appeals functionality. Model parameters 1012 stores or provides the parameters or coefficients of corresponding ones of machine learning models. During training, these parameters 1012 are adapted based on the input-output training pairs of the training data sets 1022. After the parameters 1012 are adapted (after training), the parameters are used by trained models 1060 to implement the trained machine learning models on a new set of data 1070.

Training data 1020 includes constraints 1026, which may define the constraints of a given patient information feature or a given benefit plan features. The paired training data sets 1022 may include sets of input-output pairs, such as pairs of a plurality of patient information features and features of inquiries associated with the patient information. Some components of training input 1010 may be stored separately at a different off-site facility or facility than other components.

Machine learning model(s) training 1030 trains one or more machine learning techniques based on the sets of input-output pairs of paired training data sets 1022. For example, model training 1030 may train the machine learning (ML) model parameters 1012 by minimizing a loss function based on one or more ground-truth data.

The ML models can include any one or combination of classifiers, LLMs, or neural networks, such as an artificial neural network, a convolutional neural network, an adversarial network, a generative adversarial network, a deep feed-forward network, a radial basis network, a recurrent neural network, a long/short term memory network, a gated recurrent unit, an autoencoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, a first ML model of the ML models can be applied to a training batch of patient information features to estimate or generate a prediction of inquiries associated with the prior authorization. In some implementations, a derivative of a loss function is computed based on a comparison of the estimated prediction of the prior authorization inquiries and the ground truth resulting from those inquiries, and parameters of the first ML model are updated based on the computed derivative of the loss function. The result of minimizing the loss function for multiple sets of training data trains adapts, or optimizes the model parameters 1012 of the corresponding first ML model. In this way, the first ML model is trained to establish a relationship between a plurality of training patient information and ground-truth results. This system can be repeated for each type of prior authorization that may be requested. The prior authorization available may change for each individual, each plan covering the individual, data from the provider requesting the prior authorization, and the medical condition experienced by the individual.

A second ML model of the ML models can be trained to select the correct ML model to be used in the prior authorization selection. The second ML model can access data on which the first models work, the success of the model in correctly conduct a prior authorization review, and various other patient parameters. The second ML model can be used to generate a selection system or AI bot to select the correct prior authorization model.

After the machine learning models are trained, new data 1070, including all the features related to prior authorization decisions, are received and/or derived by the prior authorization platform 1000. The first trained machine learning model may be applied to the new data 1070 to generate results 1080, including a prediction of automated prior authorization decisions. The prompts are applied to the second trained machine learning model to perform tasks for evaluation and selection of prior authorization models.

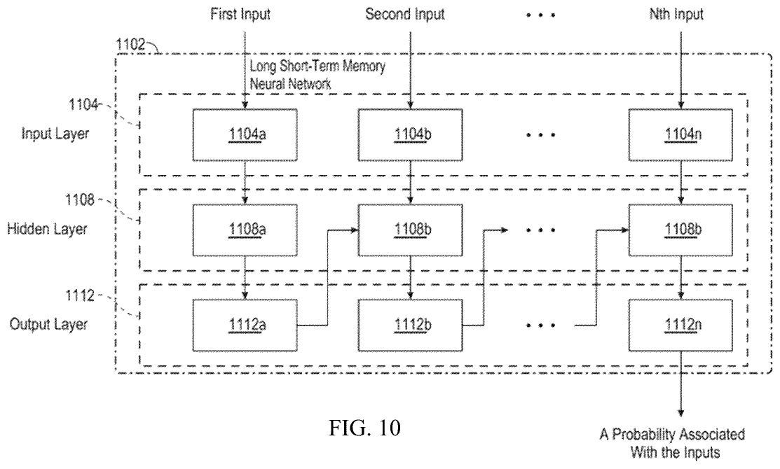

FIG. 10 is a functional block diagram of an example neural network 1102 that can be used for the inference engine or other functions (e.g., engines) as described herein to produce a predictive model. The predictive model can identify or generate inquiries associated with patient information. In an example, the neural network 1102 can be a LSTM neural network. In an example, the neural network 1102 can be a recurrent neural network (RNN). The example neural network 1102 may be used to implement the machine learning as described herein, and various implementations may use other types of machine learning networks. The neural network 1102 includes an input layer 1104, a hidden layer 1108, and an output layer 1112. The input layer 1104 includes inputs 1104a, 1104b . . . 1104n. The hidden layer 1108 includes neurons 1108a, 1108b . . . 1108n. The output layer 1112 includes outputs 1112a, 1112b . . . 1112n.

Each neuron of the hidden layer 1108 receives an input from the input layer 1104 and outputs a value to the corresponding output in the output layer 1112. For example, the neuron 1108a receives an input from the input 1104a and outputs a value to the output 1112a. Each neuron, other than the neuron 1108a, also receives an output of a previous neuron as an input. For example, the neuron 1108b receives inputs from the input 1104b and the output 1112a. In this way the output of each neuron is fed forward to the next neuron in the hidden layer 1108. The last output 1112n in the output layer 1112 outputs a probability associated with the inputs 1104a-1104n. Although the input layer 1104, the hidden layer 1108, and the output layer 1112 are depicted as each including three elements, each layer may contain any number of elements. Neurons can include one or more adjustable parameters, weights, rules, criteria, or the like.

In various implementations, each layer of the neural network 1102 must include the same number of elements as each of the other layers of the neural network 1102. For example, training features (e.g., collection of patient information associated with a first set of ground truth inquiries) may be processed to create the inputs 1104a-1104n.

The neural network 1102 may implement a first model to produce a set of inquiries. More specifically, the inputs 1104a-1104n can include fields of the patient information as data features (binary, vectors, factors or the like) stored in the storage device 110. The features of the patient information can be provided to neurons 1108a-1108n for analysis and connections between the target columns, the data on which the prior authorization is based, which model should be used for a particular medical condition, or the performance of the models individually. The neurons 1108a-1108n, upon finding connections, provides the potential connections as outputs to the output layer 1112, which determines a set of inquiries associated with the patient information.

The neural network 1102 can perform any of the above calculations. The output of the neural network 1102 can be used to control an LLM to retrieve the appropriate set of medical information. In some examples, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 1104a is connected to each of neurons 1108a, 1108b . . . 1108n.

The methods and systems herein can include the accuracy rate comprising a determination whether one of the plurality of artificial intelligence models, machine learning models, and/or large language models correctly predicted the known target column value, and wherein the confusion metric indicates whether an incorrectly predicted known target columns included more false positives or more false negatives.

In some embodiments, each of the predictive models and/or machine learning algorithms can make a prediction as to prior authorization status (e.g., “approved” or “denied” or “suspended”). By predicting the prior authorization status, each of the multiple models and/or machine learning algorithms can automatically authorize prescription claims (or actually filling prescription orders) or medical procedure claims (or actually triggering medical care), thereby saving time for physicians, pharmacies, and patients in filling a prescription or receiving care. The system can use prior decisions to establish a base model (machine learning model, neural network model or large language model). The base model can be modified based on current decisions that arise after the model was last updated. The model can identify the missing data or basis for a denial and automatically request the missing information before a decision is made and place a claim record in a “suspended” status awaiting the requested data. The model can further highlight why a claim record should be tagged with a “denied” tag. The model can further extract all of the data needed for the “approved” tag and place it in a dynamic electronic display screen for review. When a further review, e.g., of the claim record shown on a display screen, results in a change of status, the basis for the change in status is fed back into the training data for the model. The model is then updated. The tags of the status of a claim can be stored in the databases described herein.

Embodiments described herein for updating the model can adjust the word embeddings of data included in the documentation record supporting the electronic appeal request. Feature vectors for a question associated with criteria related to a specific type of authorization can be automatically updated in the model. The number of nearest neighbors of the word embeddings to the feature vector can be automatically updated in the model. The prompt for a generative artificial intelligence large language model using text from the question and text from the predetermined number of nearest neighbors can be automatically updated.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example methods and systems for using machine learning algorithms are described. In the following description, for purposes of explanation, numerous specific details are set forth in order to provide a thorough understanding of example embodiments. It will be evident, however, to one of ordinary skill in the art that embodiments of the present disclosure may be practiced without these specific details.

1. A method comprising:
a data extractor subsystem receiving an appeal request and documentation supporting the appeal request;
the data extractor subsystem creating word embeddings of data included in the documentation supporting the appeal request;
the data extractor subsystem generating a feature vector for a question associated with a criterion;
the data extractor subsystem finding a number of nearest neighbors of the word embeddings to the feature vector;
the data extractor subsystem generating a prompt for a generative artificial intelligence large language model using text from the question and text from the number of nearest neighbors;
a generative artificial intelligence subsystem implementing the generative artificial intelligence large language model generating an answer to the prompt; and
a prediction subsystem predicting a prior authorization status associated with the appeal request using the answer from the generative artificial intelligence large language model as an input to a machine learning model.

2. The method of claim 1, wherein the data extractor subsystem is further configured to concatenate the text associated with the number of nearest neighbors.

3. The method of claim 1, wherein the data extractor subsystem implements a retrieval-augmented generation method for prompt engineering.

4. The method of claim 1, wherein the word embeddings are stored in as vectors in a vector database.

5. The method of claim 1, wherein the generative artificial intelligence large language model is a large language model.

6. The method of claim 1, wherein predicting the prior authorization status associated with the appeal request using the answer from the generative artificial intelligence large language model as an input to a machine learning algorithm further comprises:
an optimization subsystem optimizing each of a plurality of deep learning/machine learning models by at least addressing class imbalances among a first subset of historical records;
the optimization subsystem training each of the plurality of deep learning/machine learning models using the first subset of historical records, wherein training each of the plurality of deep learning/machine learning models comprises each of the plurality of deep learning/machine learning models respectively analyzing each of the first subset of the historical records to determine a relationship between each target column of the respective historical records in the first subset of historical records and respective predictor columns of the respective historical records in the first subset of historical records;
the optimization subsystem implementing each of the plurality of deep learning/machine learning models, as trained, to predict a value of respective known target columns in each of a second subset of historical records based on respective predictor columns in each of the second subset of historical records;
a selection subsystem determining respective success rates for each of the plurality of deep learning/machine learning models at predicting the respective known target columns for each of the second subset of historical records, wherein each success rate comprises an accuracy rate and a confusion metric of each of the plurality of deep learning/machine learning models at predicting the respective known target columns for each of the second subset of historical records; and
the selection subsystem selecting a first of the plurality of deep learning/machine learning models having a highest success rate.

7. The method of claim 6, wherein the prediction subsystem predicting a prior authorization status associated with the appeal request using the answer from the generative artificial intelligence large language model further comprises the prediction subsystem implementing the first of the plurality of deep learning/machine learning models to decide a target column of a received authorization request based on predictor columns in the received authorization request associated with the received appeal request.

8. The method of claim 7, further comprising:
training first of the plurality of deep learning/machine learning models using the word embeddings as an additional predictor column when the first of the plurality of deep learning/machine learning models predicts that the target column should be a denial of the received authorization request.

9. The method of claim 6 wherein the plurality of deep learning/machine learning models comprises a feed-forward neural network, a convolutional neural network, a recurrent neural network, a Random Forest model, a Naïve Bayes model, a decision trees model, and a logistic regression model.

10. The method of claim 1 further comprising communicating the prediction to one or more stakeholders.

11. The method of claim 1, wherein the data extractor subsystem uses cosine similarity, Euclidean distance, cosine similarity, or dot product to determine the number of nearest neighbor word embeddings.

12. A system for automatic processing prescription, comprising:
a storage device to store a plurality of machine learning algorithms; and
a subsystem in communication with the storage device and configured to:
receive an appeal request and documentation supporting the appeal request;
create word embeddings of data included in the documentation supporting the appeal request;
generate a feature vector for a question associated with a criterion;
find a number of nearest neighbors of the word embeddings to the feature vector;
generate a prompt for a generative artificial intelligence large language model using text from the question and text from the number of nearest neighbors;
generate an answer to the prompt using the generative artificial intelligence large language model; and
predict a prior authorization status associated with the appeal request using the answer from the generative artificial intelligence large language model as an input to a machine learning model.

13. The system of claim 12, wherein the subsystem is further configured to concatenate the text associated with the number of nearest neighbors.

14. The system of claim 12, wherein the subsystem implements a retrieval-augmented generation method for prompt engineering.

15. The system of claim 12, wherein the word embeddings are stored in as vectors in a vector database.

16. The system of claim 12, wherein the generative artificial intelligence large language model is a large language model.

17. The system of claim 12, wherein the subsystem predicts the prior authorization status by being further configured to:
optimize each of a plurality of deep learning/machine learning models by at least addressing class imbalances among a first subset of historical records;
train each of the plurality of deep learning/machine learning models using the first subset of historical records, wherein training each of the plurality of deep learning/machine learning models comprises each of the plurality of deep learning/machine learning models respectively analyzing each of the first subset of the historical records to determine a relationship between each target column of the respective historical records in the first subset of historical records and respective predictor columns of the respective historical records in the first subset of historical records;
implement each of the plurality of deep learning/machine learning models, as trained, to predict the value of respective known target columns in each of a second subset of historical records based on respective predictor columns in each of the second subset of historical records;
determine respective success rates for each of the plurality of deep learning/machine learning models at predicting the respective known target columns for each of the second subset of historical records, wherein each success rate comprises an accuracy rate and a confusion metric of each of the plurality of deep learning/machine learning models at predicting the respective known target columns for each of the second subset of historical records; and
select a first of the plurality of deep learning/machine learning models having a highest success rate.

18. The system of claim 17, wherein the subsystem is further configured to implement the first of the plurality of deep learning/machine learning models to decide a target column of a received authorization request based on predictor columns in the received authorization request associated with the received appeal request.

19. The method of claim 18, wherein the subsystem is further configured to train the first of the plurality of deep learning/machine learning models using the word embeddings as an additional predictor column when the first of the plurality of deep learning/machine learning models predicts that the target column should be a denial of the received authorization request.

20. The system of claim 16 wherein the plurality of deep learning/machine learning models comprises a feed-forward neural network, a convolutional neural network, a recurrent neural network, a Random Forest model, a Naïve Bayes model, a decision trees model, and a logistic regression model.

21. The system of claim 12, wherein the subsystem uses cosine similarity, Euclidean distance, cosine similarity, or dot product to determine the predetermined number of nearest neighbor word embeddings.

22. A non-transitory machine-readable medium comprising instructions, which, when executed by one or more processors, cause the one or more processors to perform the following operations:
receive an appeal request and documentation supporting the appeal request;
create word embeddings of data included in the documentation supporting the appeal request;
generate a feature vector for a question associated with a criterion;
find a number of nearest neighbors of the word embeddings to the feature vector;
generate a prompt for a generative artificial intelligence large language model using text from the question and text from the number of nearest neighbors;
generate an answer to the prompt using the generative artificial intelligence large language model;
predict a prior authorization status associated with the appeal request using the answer from the generative artificial intelligence large language model as an input to a machine learning model; and
train the machine learning model using the word embeddings as an additional predictor column when the first of the plurality of deep learning/machine learning models predicts that a target column should be a denial of the prior authorization status associated with the appeal request.
